(19) United States
(12) Patent Application Publication
(10) Pub. No.: US 2010/0062426 A1
(43) Pub. Date: Mar. 11, 2010

(54) AGR2 and TFF3 Regulation in the Diagnosis and Treatment of Cancer

(76) Inventors: Shawn Mark O'Hara, Ambler, PA (US); Denis Smimov, Media, PA (US); Daniel R. Zweitzig, Feasterville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/092,626

(22) PCT Filed: Nov. 3, 2005
(86) PCT No.: PCT/US05/39602

§ 371 Date: Oct. 16, 2009

(51) Int. Cl. C12Q 1/68

(52) U.S. Cl. 435/6

(57) ABSTRACT

A method for assessing tumor progression is described by assessing AGR2 and/or TFF3 expression in a biological sample after induction of a physiological stress, such as hypoxia or serum deprivation in an enriched sample. Assessing the role of these indicators and their expression levels in an enriched CTC sample provides diagnostic and prognositic information on a patient. This method is also useful as a pharmatool in drug discovery.

### BACKGROUND OF THE INVENTION

1. Field of the Invention

This invention relates generally to gene specific amplification, analysis and profiling of cytosolic biomolecules useful in the fields of oncology, diagnostic testing and pharmacogenomics (personalized medicine). The invention is particularly useful in such fields as cancer screening, selecting (identification and stratification of therapy responders/non-responders) and monitoring for chemotherapy treatment, or cancer recurrence.

2. Description of Related Art

The ability of tumor cells to metastasize to distant organs is responsible for most cancer deaths. In recent years, several studies have used gene expression profiling analysis on primary and metastatic tumors resulting in identification of genes having potential roles in tumor progression towards metastasis. However, despite recent advances, the molecular signaling mechanisms associated with metastasis remain poorly understood. Detection and characterization of disseminated tumor cells is beginning to aid in the dissection of the metastatic cascade, or the different events that lead to primary and secondary metastases in patients with cancer. In fact, enumeration of circulating tumor cells (CTCs) has recently been shown to be an independent predictor of progression-free survival and overall survival in patients with metastatic breast cancer (Cristofanilli, M., et al., 2004. Circulating Tumor Cells, Disease Progression, and Survival in Metastatic Breast Cancer. N. Eng. Jour Med 351, 781-791).

In addition to enumeration, many studies have been published regarding gene expression profiling of CTCs. These studies have yielded useful clinical information, but are limited to the evaluation of genes previously identified in solid tumors (O'Hara S M, et al., 2004. Multigene Reverse Transcription-PCR Profiling of Circulating Tumor Cells in Hormone-Refractory Prostate Cancer. Clin Chem. 50, 826-35.). Characterization of novel signaling mechanisms required for expression of genes prevalently expressed in CTCs will be crucial to gaining insight into the multi-step processes of tumor progression towards metastasis and should aid in the design of more targeted therapies.

A major characteristic required of metastatic cells is the ability to adapt to and survive the insults of pathophysiological stress before, during and after dissemination. Successful metastasis most likely requires the de novo expression, or activation of genes that augment survival of tumor cells during periods of pathophysiological stress such as hypoxia, loss of exogenous to growth factors, oxidative stress, immune response. Consequently, much research has been focused on characterizing genes involved in mediating the adaptive responses of tumor cells during periods of stress such as hypoxia resulting from a local decrease in blood supply. Similar studies have also associated the ability of tumor cells to survive and proliferate under hypoxic conditions with poor prognosis and resistance to radiation therapy. It is not surprising then that the molecular mechanisms associated with tumor cell survival during hypoxic conditions are now being targeted for novel therapeutic agents. Thus we feel it is important to identify and characterize clinically relevant metastatic gene markers induced in response to physiological stress.

Since breast cancer cells are particularly well known to adapt and survive periods of stress such as hypoxia (Knowles, et al., 2001. Hypoxia and Oxidative Stress in Breast Cancer: Hypoxia and tumourigenisis. Br Can Res 3, 318-322; Pugh, et al., 2001. Hypoxia and Oxidative Stress in Breast Cancer: Hypoxia Signalling Pathways. Br Can Res 3, 313-317) accompanied by serum deprivation, we developed a method to monitor the expression of a panel of genes identified as breast CTC identification markers, during exposure of breast cancer cell lines to hypoxia, serum deprivation and a combination thereof. Our invention reveals that serum deprivation alone, and especially serum deprivation in combination with hypoxia, lead to a dramatic increase in human anterior gradient-2 (Hag-2, AGR2) and intestinal trefoil factor-3 (TFF3, ITF3) mRNA expression. This invention provides a method and means into how CTC markers are regulated in vivo and could ultimately aid in the design of novel therapies targeted at blocking breast cancer metastasis.

### SUMMARY OF THE INVENTION

The present invention provides a method and means for diagnosing cancer by utilizing the role of AGR2 and TFF3 metabolism to physiological stress. Tumors from breast cancer patients express higher levels of these genes when compared to normal tissue when subjected to stress. After normalization to ubiquitin, AGR2 and TFF3 expression increases (approximately 60%) of patient matched tumor samples when compared to normal tissue. This increase provides the foundation for assessing disease state, response to therapy and other prognostic values. The method is applicable in cancers with overexpression of one or both genes such as ovarian, lung and thyroid tumors, colon, stomach, rectum and prostate in an immunomagnetically enriched sample. Accordingly, breast cancer cells co-adapt the use of AGR2 and TFF3 to mediate cell survival and repair, similar to their role in normal intestinal epithelial cells.

### BRIEF DESCRIPTION OF THE DRAWINGS

FIG. 1 shows the expression levels after induction to stress. Panel A and B depict the induction of AGR2 and TFF3 expression, respectively. Ca induction of VEGF is shown in panel C. Induction of S100A16 is shown in panel D.

FIG. 2 Serum deprivation of MDA-MB-231 cells are shown. Panel A shows AGR2 induction blocked with ERK1/2 after both serum deprivation and hypoxia. Panel B shows inhibition of TFF3 was only inhibited after serum deprivation. Panel C and D show a lack of inhibition with VEGF induction and hypoxia.

FIG. 3 expression levels of AGR2 and TFF3 after normalization to ubiquitin. A standard cancer blot was used to assess expression levels in multiple tumor samples. Each sample shows an upregulation of the tumor sample compared with their mateched healthy tissue.

### DETAILED DESCRIPTION OF THE PREFERRED EMBODIMENTS

The ability of tumor cells to metastasize to distant organs is responsible for most cancer deaths. Despite a growing amount of research, the molecular mechanisms associated with tumor progression towards metastasis remain poorly understood. In recent years, much of the research on this subject has been initiated on genes identified by techniques such as microarray analyses and proteomic profiling of tumor tissues and cell lines. These types of studies are, and will continue to be, crucial to gaining insight into the multi-step processes of tumor progression. Anterior gradient 2 (AGR2) is a recently to discovered human homologue of the secreted Xenopus laevis protein XAG-2. XAG-2 is expressed in the cement gland of Xenopus laevis and is associated with anteroposterior fate determination during early development. Sequence analysis of AGR2 revealed a predicted N-terminal cleavable secretory signal that suggests it is a secreted protein in humans as well. An increased interest in AGR2 was derived from the original finding that it is co-expressed with the estrogen receptor in breast cancer cell lines. AGR2 expression in an enriched sample of circulating tumor cells derived from breast, prostate and colon cancer patients would provide a diagnostic/prognostic tool in assessing these disease states. Thus making AGR2 a clinically relevant marker in cancer progression.

### Induction of AGR2 and TFF3 During Stress Treatment.

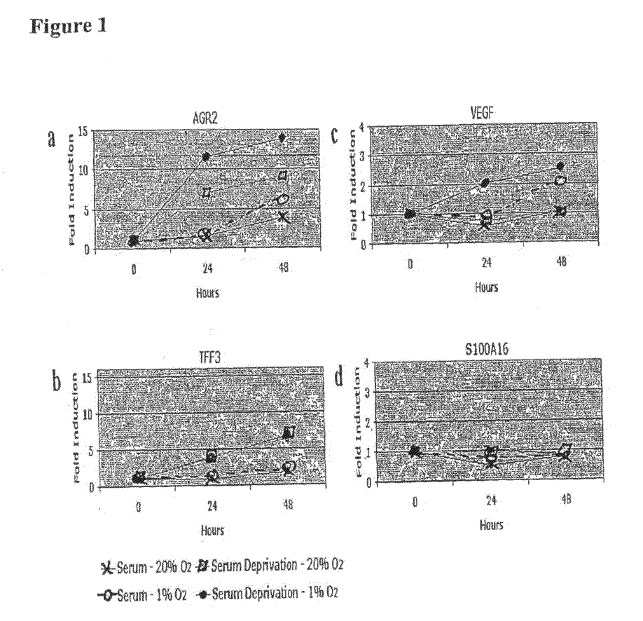

Breast cancer lines are subjected to serum starvation, hypoxia and a combination thereof. These treatments mimic conditions experienced by breast tumors during periods of pathophysiological stress resulting from decreased blood supply. Expression levels of genes, previously identified to be potential candidates for breast cancer CTC detection are measured by quantitative RT-PCR before and after stress induction for comparison. Serum deprivation alone, and especially serum deprivation in combination with hypoxia, leads to a dramatic increase in the expression of anterior gradient-2 (AGR2) and trefoil factor 3 (TFF3, ITF3) in breast cancer cells. The most dramatic induction of AGR2 and TFF3 expression is observed in the MDA-MB-231 cell line (FIGS. 1A and B). Co-induction of the classic hypoxia responding gene VEGF suggests that the hypoxia response pathway is activated during treatment of cells (FIG. 1C). In addition to these genes, the expression of S100A16 is monitored to verify the observed induction of AGR2 and TFF3 which is a specific response to the applied stress conditions (FIG. 1D). After repeating stress treatment of MDA-MB-231 cells, similar levels of induction for HAG-2 and TFF3 are observed. These results, along with prevalent expression of HAG-2 and TFF3 in CTCs, suggest that AGR2 and TFF3 play an important role in a cellular response to hostile growth conditions experienced by tumor cells before and after dissemination. Thus, there is a suggestion that both AGR2 and TFF3 play a role in breast cancer cell survival during periods of physiological stress.

### Er1/2 Pathway is Involved in the Activation of AGR2 and TFF3 Transcription During Stress Treatment.

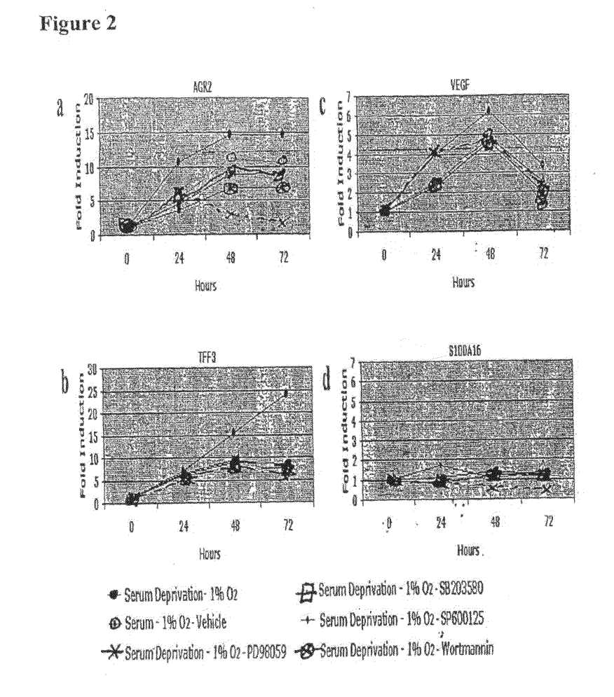

Chemical inhibitors of ERK1/2, JNK, p38 and PI3K were used in an attempt to better understand the signaling pathways responsible for induction is of AGR2 and TFF3 during serum deprivation and hypoxic treatment of MDA-MB-231 cells. Cells were treated with serum deprivation, hypoxia and a combination thereof for 48 hours in the presence and absence of each inhibitor. After treatment and quantitative PCR analysis, we observed that the ERK1/2 inhibitor, PD98059, was sufficient to block induction of AGR2 by serum deprivation alone and its combination with hypoxia (FIG. 2 A). In contrast, TFF3 induction was blocked by PD98059 only during treatment with serum deprivation. Interestingly, induction of TFF3 was not inhibited when serum deprivation was combined with hypoxia (FIG. 2 B), suggesting alternative-signaling mechanisms are responsible for activation of this gene in response to different stimuli. VEGF induction and S100A16 expression were also not affected by treatment with inhibitors, further suggesting that the effect of PD98059 is specific for blocking AGR2 induction (FIGS. 2 C and D).

### Overexpression of AGR2 and TFF3 in Breast Tumors.

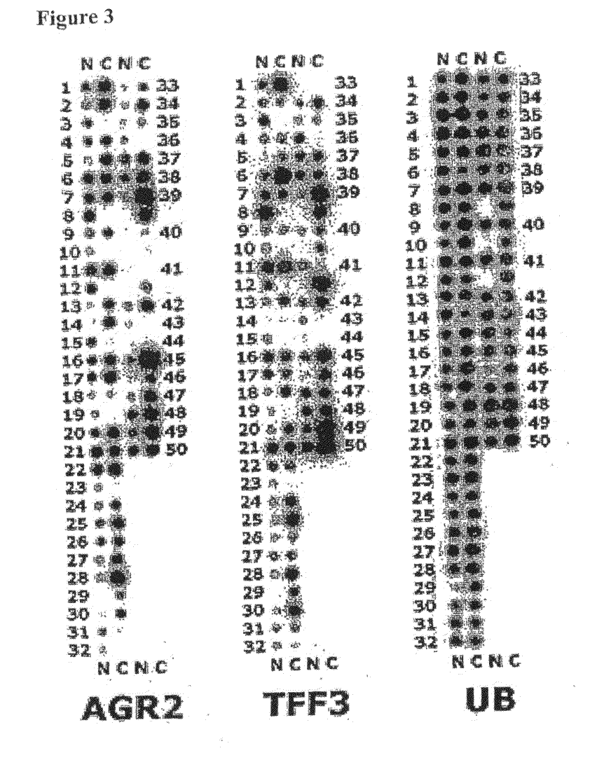

Because AGR2 and TFF3 play a significant role in the response of breast cancer cells to physiological stress, it to be advantageous for breast tumors to express higher levels of these genes when compared to normal tissue. Using a commercially available cancer-profiling array to compare the expression of AGR2 and TFF3 in patient matched normal and breast cancer samples, AGR2 and TFF3 expression increases in approximately 60% of patient matched tumor samples when compared to normal tissue and after normalization to ubiquitin (FIG. 3). The over-expression in breast tumors suggests a role in progression towards metastasis. Thus, during physiological stress, breast cancer cells co-adapt the use of AGR2 and TFF3 to mediate cell survival and repair, similar to their role in normal intestinal epithelial cells.

The present invention combines immunomagnetic enrichment of patient samples as discussed in U.S. Pat. No. 6,365,362 and U.S. Pat. No. 6,645,731 (both incorporated by reference) with a stress-induced induction of AGR2 and TFF3 to provide a method in cancer diagnosis.

We claim:

1. A method for detecting and enumerating circulating tumor cells in a mixed cell population, the presence of said cells in said population being indicative of a disease state, comprising:
a) preparing an immunomagnetic sample wherein a biological specimen from a test subject, which specimen comprises a mixed cell population suspected of containing said CTC cells, which CTC cells are present at 1 to 50 cells per ml, is mixed with magnetic particles coupled to a biospecific ligand which reacts specifically with the CTC cells, to the substantial exclusion of other sample components;
b) inducing a physiological stress on the CTC cells;
c) determining the expression levels of a gene from a group consisting of AGR2, TFF3, and combinations thereof;
d) analyzing said genes and CTC cells wherein the greater the number of CTC cells expressing high levels of said gene the greater the severity of said disease state.

2. A method as claimed in claim 1, wherein as an intermediate step between the preparation of the immunomagnetic sample and inducing a physiological stress, said immunomagnetic sample is subjected to a magnetic field to produce an enriched CTC cell suspension as the immunomagnetic sample.

3. A method as claimed in claim 1, wherein said disease state is cancer.

4. A method as claimed in claim 1, wherein said biospecific ligand is a monoclonal antibody specific for at least one cancer cell determinant.

5. A method as claimed in claim 1, wherein said method further comprises the step of assessing the malignant status of the labeled cancer cell-containing fraction by immunocytochemical analysis.

6. A method as claimed in claim 1, wherein said biological specimen is obtained from said test subject periodically and assessed for the presence and number of circulating cancer cells as an indicator of either progression of said disease state, or the patient's response to cancer eradication procedures.

7. A method as claimed in claim 1, wherein said biospecific ligand binds specifically to an epithelial cell adhesion molecule.

8. A method as claimed in any of claims 1, wherein said biological specimen is peripheral blood

9. The method according to claim 3 wherein the cancer or carcinoma is selected from the group consisting of prostate cancer, breast cancer, colon cancer apudoma, choristoma, branchioma, malignant carcinoid syndrome, carcinoid heart disease, carcinoma e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell and transitional cell reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, throphoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, antiokeratoma, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (Kaposi's, and mast-cell), neoplasms (e.g., bone, digestive system, liver, pancreatic, pituitary, testicular, orbital, head and neck, central nervous system, acoustic, pelvic, respiratory tract, and urogenital), neurofibromatosis, and cervical dysplasia.

10. A method as claimed in claim 9 wherein the carcinoma is a breast carcinoma.
